(19) United States
(12) Patent Application Publication
(10) Pub. No.: US 2021/0150348 A1
(43) Pub. Date: May 20, 2021

(54) MODEL OF FAST-SPIKING NEURONS REGULATING NEURAL NETWORKS

(71) Applicant: Patricia MacKenzie, San Jose, CA (US)

(72) Inventor: Patricia MacKenzie, San Jose, CA (US)

(21) Appl. No.: 17/098,435

(22) Filed: Nov. 15, 2020

Related U.S. Application Data
(60) Provisional application No. 62/935,659, filed on Nov. 15, 2019.
(60) Provisional application No. 63/048,615, filed on Jul. 6, 2020.

(51) Int. Cl. G06N 3/08; G06N 3/04; G06N 3/063; A61B 5/31; A61B 5/384; A61B 5/00

(52) U.S. Cl. CPC G06N 3/08; G06N 3/049; A61B 5/6831; A61B 5/31; A61B 5/384; G06N 3/063

(57) ABSTRACT

A model of neural networks comprised of the fast-spiking class of interneurons regulating neural networks. Fast-spiking neurons regulate activity in neural networks in response to environmental input from thalamic afferents by providing strong, rapid inhibition to a plurality of neurons in advance of excitatory neurons responding to environmental input. Fast-spiking neurons regulate experience dependent plasticity in neural networks by shifting between distinct maturational states in response to experience.

### BACKGROUND

Artificial intelligence (AI) is often based on human intelligence. The connectivity of a convolutional neural network (CNN), for example, is modeled after the connectivity of biological neural networks that perceive visual information. Neurons in the human retina are spatially organized according to receptive fields that comprise the visual information they respond to. Receptive fields overlap such that the entire field of vision is represented. Receptive fields span synaptic connections, meaning the receptive field of a photoreceptor is retained in the spatial connectivity of a postsynaptic retinal ganglion cell and thalamic neurons innervating the visual cortex. CNNs use spatial organization and similar layers of connectivity as circuitry of the retina; each layer performs a calculation and outputs that information to the next layer and neurons may pool information between layers.

Recent advancements in combining CNNs with the computational power provided by high performance CPUs and GPUs has enabled AI to outperform humans at simple image recognition tasks. The advancement of AI informed by biological neural networks is restricted by lack of understanding of biological neural networks. Lack of understanding of biological neural networks poses an additional challenge, in that, it is difficult to predict how AI, and more generally technology, will influence the brain. Technology does not necessarily augment human health and ability and may pose a negative impact on mental health and well-being. In the same vein, treatments for neurological disorders are not always advantageous. For example, pharmacological agents that ameliorate neurological disorders by targeting a molecular pathway or receptor may cause paradoxical effects and decrease cognitive performance.

### SUMMARY

This specification relates a model of how biological neural networks change. In this model, the fast-spiking (FS) class of inhibitory cortical neurons regulates activity in neural networks in response to experience. FS neuron activity may be used to identify which neural networks are active in response to an experience and how neural network activity conifers cognitive abilities. FS neuron mediated inhibition onto a plurality of connected excitatory neurons enables precise regulation of temporal and spatial activity in neural networks. Excitatory neurons receive comparatively weaker and broader input from the environment and strong inhibitory input from FS neurons. FS regulation of neural networks enables regulation of synaptic plasticity by coordinating the timing of activity, enabling FS regulation to be used to predict changes in neural networks.

FS neuron mediated regulation of activity enables local regulation of experience dependent plasticity, a biological process in which experience is reflected in the structure and function of cortical neural networks. FS neurons regulate experience dependent plasticity by shifting between maturation states in response to experience, with each state corresponding to distinct FS neuron properties and shifts between states enabling remodeling FS neural connectivity, which enables neural networks to encode a new experiences. Identification of FS regulation of experience dependent plasticity through identifying shifts in FS maturation states enables the elucidation of the structure of neural networks and the cognitive abilities and the associated memories reflected by an experience. Understanding of the structure and function of neural networks may be used to advance AI, which is based on models of biological intelligence.

Experience dependent plasticity is correlated to vulnerability to myriad neurological disorders and increased cognitive abilities. Understanding neural networks allows for predictions relating to FS regulation of neural networks that may be used to provide recommendations for experience and therapeutic agents which may treat neurological disorders and augment cognitive abilities. A method is provided for establishing a model of FS regulation of neural networks that may be integrated with biological neural networks on a brain recording wearable device that may be worn as a wristband.

The details of one or more embodiments of the subject matter of this specification are set forth in the accompanying drawings and the description below. Other features, aspects, and advantages of the subject matter will become apparent from the description and accompanying drawings, wherein:

### DESCRIPTION OF DRAWINGS

FIG. 1 is a model wherein FS neurons regulate activity in neural networks;

FIG. 2 is a model wherein FS neurons regulate experience dependent plasticity in neural networks;

FIG. 3 illustrates FS maturation states;

FIG. 4 illustrates activity dependent regulation of gene expression in FS neurons;

FIG. 5 is a method of establishing a model wherein FS neurons regulate activity in neural networks;

FIG. 6 is a method of establishing a model wherein FS neurons regulate experience dependent plasticity in neural networks;

FIG. 7 illustrates how this model may be used to elucidate neural network structure and function;

FIG. 8 illustrates how this model may be used to shift neural networks;

FIG. 9 is a representative example wherein this model is established using a brain recording wearable device;

FIG. 10 is an example of a brain recording wearable device;

FIG. 11 illustrates the brain recording wearable device being worn on the wrist;

FIG. 12 is an outer view of the brain recording wearable device;

FIG. 13 is an inner view of the brain recording wearable device;

FIG. 14 is a view of the inside of the brain recording wearable device;

FIG. 15 is a circuit diagram of components of the brain recording wearable device.

### DETAILED DESCRIPTION

It is to be understood by those of ordinary skill in the art that, although myriad details are set forth in order to aid understanding of the embodiments described herein, these embodiments may be practiced without these specific details. In other instances, well-known methods, procedures and components have not been described in detail so as not to obscure the embodiments described herein. This description is not to be considered as limiting the scope of the embodiments described herein.

As would be known by a skilled practitioner, any processor, computer, device, module, unit, component, or server that executes instructions may include or otherwise have access to computer readable media, which may be storage media, computer storage media, or data storage devices (removable and/or non-removable). Computer storage media may include volatile and non-volatile, removable and non-removable media implemented in any method or technology for storage of information, such as computer readable instructions, data structures, program modules, or other data. Any such computer storage media may be part of the device or accessible or connectable to a device. The applications and models detailed in this disclosure may be implemented using computer readable/executable instructions that may be stored or otherwise held by such computer readable media.

Biological neural networks are comprised of neurons that are connected electrochemically through synapses or gap junctions. In biological neural networks, this definition may be expanded to additional forms of connectivity, including and not limited to small molecules like growth factors released across synapses and additional cell types, including and not limited to microglia. It should be understood that biological neural networks are comprised of very large numbers of neurons with myriad complex configurations and the models provided are anatomically representative illustrations. In biological neural networks, connection weight is defined by the change in resting membrane potential caused by opening receptors at a synapse; weight reflects contribution of a synaptic event to the probability of a neuron firing an action potential. Activity is typically defined as a neuron firing one or more action potentials and may include electrical activity at gap junctions. Activity may include electrochemical communication that occurs at synapses, and is further specified as synaptic activity for clarity.

Biological neural networks inform the architecture of artificial intelligence (AI), including but not limited to artificial neural networks and bayesian and other graphical models. The spatial and temporal connectivity of known biological neural networks have informed myriad computational models, with variations in the mathematical equations performed by a model. Similarly, applying mathematic equations describing biologically accurate neural communication to models of biological neural networks with known properties is a common approach in computational neuroscience. As would be understand by a skilled practitioner, the following principles generally apply to the design of AI and other forms of computational models based on biological neural networks.

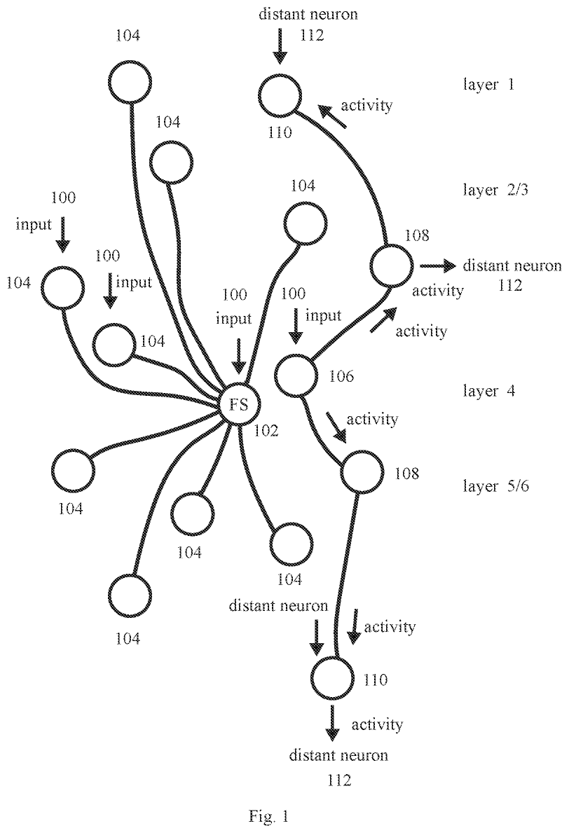

Neural networks in the cerebral cortex (cortex) likely conifer the cognitive abilities that make us human, as cortex is an evolutionarily new structure. In reference to FIG. 1, fast-spiking (FS) interneurons in the cortex regulate the activity of neurons in a biological neural network. FS neurons are nonsomatostatin expressing cells of medial ganglionic eminence origin and may be identified throughout postnatal development in mice using line G42 (GAD67-EGFP transgenic strain G42). In this disclosure FS neurons are defined by their developmental origin rather than the marker parvalbumin, which inconsistently labels a subset of FS neurons. FS neurons are a rare cell type; the majority of neurons in the cortex are excitatory projection neurons. FS neurons provide the majority of inhibition in the cortex, as defined by connection weight and number of connections; FS neurons make significantly more synaptic connections and FS neuron mediated synaptic events have greater connection weight and rapid kinetics in comparison to other neural cell types.

Cortical neurons receive thalamic input (100), with organization that reflects the topographical organization of information relayed from the external environment, with some thalamic affarants diffusely innervating many cortical areas. Thalamic innervation (100) of cortex retains spatial and temporal dynamics originating from the environment, relaying preprocessed sensory information to cortical neurons. FS neurons (102) receive strong and rapid synaptic input from the thalamus (100); one event at a thalamus-FS synapse is sufficient to result in an FS neuron firing one or more action potentials. A subset of excitatory neurons (104, 106), mainly layer 4/5 excitatory cells, are innervated by thalamic afferents (100). Excitatory neurons are innervated by thalamus more broadly than FS neurons, and may be connected to multiple thalamic nuclei that relay a range of sensory information. Neurons comprising cortical neural networks in different cortical areas differ in receptive field properties, as they receive distinct thalamic inputs, which is reflected in the structure and function of the neural network.

In primary auditory cortex in mice, for example, FS neurons respond to cortical input in a similar range as excitatory neurons, which receive comparably weak, broad input originating from multiple hair cells while FS neurons generally receive thalamic input from one hair cell. FS neurons locally inhibit excitatory neurons that respond to broad thalamic input and rarely inhibit excitatory neurons that respond to the same input (within ˜50 nm) and these cells are instead inhibited by neighboring FS neurons. The structure of this neural network increases acuity in auditory perception, as it enables co-tuning and lateral inhibition.

Thalamic input (100) to excitatory neurons (104, 106) is weak in comparison to thalamic-FS synaptic connections, meaning excitatory neurons require summation of multiple excitatory synaptic inputs in order to fire one or more action potentials. Cortical excitatory neurons connect to local cortical neurons (108) and make long-range connections to neurons in other brain areas (110). Excitatory neurons receiving thalamic input can integrate activity from myriad neurons and thalamus and output onto postsynaptic neurons. Excitatory neurons in both superficial and deep layers receive local input from cortical neurons and long range input and output to local neurons and long range connections, and additionally may be connected to a diversity of interneurons. FS neurons may additionally be activated by excitatory neurons, and may recurrently inhibit excitatory neurons.

Strong, rapid connection properties enable FS neurons to respond to thalamic input in advance of thalamic-excitatory cortical neurons. FS mediated synaptic events are characterized by high amplitude and rapid kinetics and perisomatic localization, which may be sufficient to prevent postsynaptic neurons from firing one or more action potentials. FS inhibition to excitatory neurons (104) prevents neurons that are weakly or indirectly activated by thalamic input to integrate and fire action potentials, restricting activity to a small number of local excitatory thalamic targets (106) that summate multiple excitatory events during a brief window of excitation.

FS neurons connect to a plurality of neurons in multiple layers of the cortex, and are thought to form 10-100 or more times as many synaptic connections as excitatory neurons. The activity of local neural networks is regulated by FS neurons through selective connectivity with nearby excitatory neurons. FS neuron mediated regulation of local, spatially defined cortical neurons enables highly synchronized firing, as FS neurons may inhibit excitatory neurons receiving thalamic input as well as local excitatory neurons connected to excitatory neurons receiving thalamic input. FS neuronal properties enable regulation of the timing of activity and which neurons are active in the network, in a manner that reflects environmental information relayed by the thalamus. FS neurons significantly restricts activity in the cortex to a small number of active neurons (106, 108, 110), which may make long range connections to other brain areas (112). While FS neurons are local regulators, regulating activity in the cortex regulates the integration of cortical neurons with distant brain areas. This network model allows for myriad variations for differing networks, as connectivity to local neurons including and not limited to other classes of interneurons and excitatory connectivity can vary within this model depending on which neurons are regulated by FS neurons and how these neurons are connected within networks that may span throughout the brain.

FS neurons may regulate synaptic plasticity by coordinating the timing of activity neurons, and coordination of timing may include but is not limited to synchronized oscillatory brain activity. FS neurons uniquely initiate high frequency oscillatory activity in the cortex due to their uniquely strong connectivity and rapid temporal properties; FS neurons are uniquely able to sustain high frequency firing, a property requisite for initiating high frequency synchronized oscillatory activity in neural networks. Coordinated brain activity may include but is not limited to synchronized high frequency activity which is initiated by FS neurons and secondary slow oscillatory activity in neural networks regulated by FS neurons. Temporally regulating activity and recurrent excitation of active excitatory neurons onto FS neurons may enable synchronized firing in cortical neurons. Synchronized cortical neurons support rhythmic firing in brain areas that cortical projection neurons synapse onto, for example hippocampal slow wave activity. Synchronizing cortical networks can regulate slower wave activity in other brain areas and cortical neurons.

Cortical development is unique in that cortical neural networks acutely change during development in response to thalamic input relayed from the environment, such that experience is reflected in neural networks, a process called experience dependent plasticity. FS neurons role as regulators enable FS neurons to regulate neural plasticity, including and not limited to experience dependent plasticity, in local cortical neural networks and the brain areas connected to these local cortical neuronal networks.

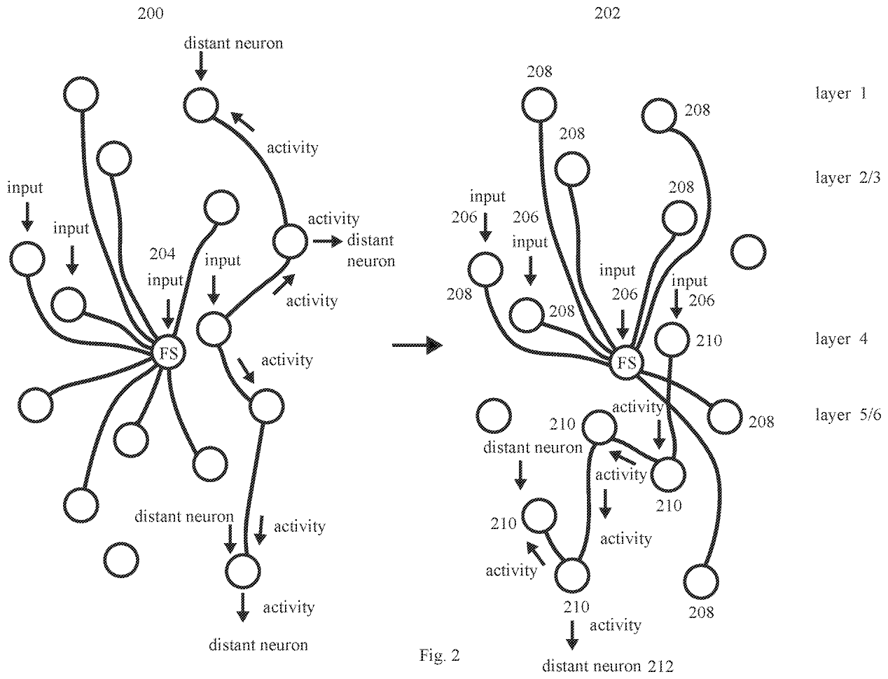

In reference to FIG. 2, FS neurons regulate experience dependent plasticity in neural networks. FS neurons regulate experience dependent plasticity by shifting between distinct maturation states in response to experience, which have been found to be distinct in a manner that is similar to cell type fate determination during neural development. FS neurons may retract and reform connections during transitions between maturation states. This is illustrated in a neural network (200) that has undergone experience dependent plasticity (202) in response to thalamic input (204). Shifting to an earlier, more plastic state initiates reformation of cortical neural networks to reflect the experience during brief time window that correlate to FS neurons retracting and reforming connections in response to thalamic input (206). FS neuron maturation is experience dependent, meaning experience relayed through the thalamus is required for FS neurons to shift to a different maturation state. FS neurons regulate the structure and function of neural networks by forming new connections (208). Redefining the inhibitory connectivity redefines which neurons are active (210) in response to experience. It has been found that changes in cortical neurons are propagated to other brain areas (212) connected to cortical neurons, for example the striatum and the hippocampus. Local neural network regulation is retained, meaning, FS neurons that are spatially nearby may be in disparate maturation states depending on thalamic input.

Experience is encoded into a neural network during transitional states, corresponding to FS neurons forming new connections and translating gene expression into functional properties. Shifts between FS maturation states occur quickly and correspond to windows wherein experience dependent plasticity occurs. Although this may require additional time, it has been found that this process may occur in 3-5 days in humans. Experience dependent plasticity in cortical neural networks was thought to be restricted to a short window during early development, however, it has been found that FS neurons may return to earlier states in adults. Returning to earlier states corresponds to learning and memory. Adult experience dependent plasticity, similarly to developmental plasticity, may change the structure and function of neural networks to reflect experience. While other cell types in the cortex do not require experience to differentiate into adult neurons and remain identifiable, changes in properties reflect changes in FS regulation properties in each state, for example, excitatory neurons are more active when FS neurons are in less mature states.

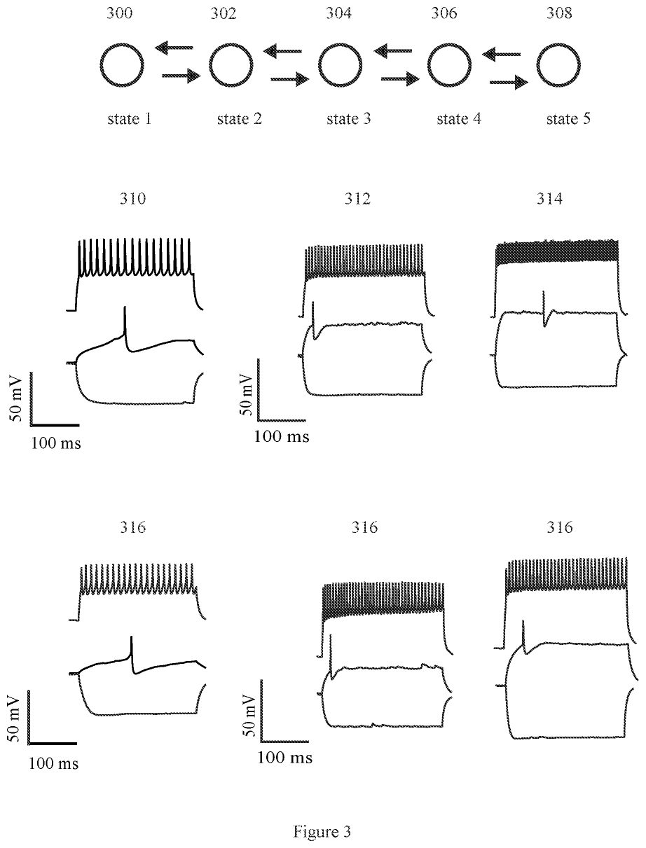

In reference to FIG. 3, the functional properties of FS neurons that define each state were initially elucidated in the auditory cortex of mice, wherein experience dependent development of FS neurons corresponds to postnatal ages (p) 12-13 (state 1), p17-18 (state 2), and postnatal ages above 21 (state 3) but not affected by senescence, with transitions between maturation states occurring between p14-16 and p19-21. Examples of neurons in state 1 (300), state 2 (302), and state 3 (304) illustrate physiological properties in each state during development and in adult FS neurons in state 1 (306), and state 2 (308). FS neurons may express the markers NK1 homeobox 2, LEVI homeobox protein 6, distal-less homeobox 1/2, visual system homeobox 2 during early development and homeobox C13 and distal-less homeobox 3 later in development. FS neural function shifts reflect transcriptional profile shifts; FS maturational shifts correspond to FS maturational expression profiles and physiological function that correspond to discrete maturation states. Developmental ages, like fate determination in early development, determine the genes generally expressed in the cell, defining the possibilities for the types of connections formed and functional properties like action potentials. As would be known by a skilled practitioner, like changes in cell fate, FS maturation state shifts mechanistically may be defined by chromatin remodeling and transcriptional regulation of gene expression. Similarly to changes in fate cell determination in early development, FS neurons may upregulate expression of growth factors and support factors for glial cell development.

It has been found that FS maturation states are retained in an identifiable way in humans and correspond to human cortical development. It should be understood that FS neuron properties are variable between individual neurons and between different spatial locations, individuals, and species. Cortex develops at varying speeds in different cortical areas. For this reason, states are defined by previously characterized physiological properties of FS regulators rather than strictly a uniform age in human development. It should be understood that myriad properties change based on states, including and not limited to inhibitory weight, localization, and connectivity, with FS neurons generally becoming faster and stronger in more mature states with increasingly spatially refined connectivity.

Coordination of network activity, synaptic plasticity and connectivity possibilities are reflected by states. The ability to initiate and sustain high frequency firing required to initiate high frequency oscillatory activity develops according to FS maturation states. Few FS neurons are able to sustain high frequency firing rates required to initiate coordinated oscillatory activity prior to experience regulated development. The maturation state of FS neurons may be determined by high frequency firing, with the probability that groups of FS neurons may fire at higher frequencies corresponding later maturational states. For example, FS maximum firing abilities characteristically change in each state, with the average being 317 Hz for maturation state 3 (p35-44, 300 Hz for p22-28), 202 Hz for maturation state 2 (p17-18) and 121 Hz for maturation state 1 (p11-13) in auditory cortex in mice. FS firing properties may be reflected in myriad properties, for example, in humans, the population of FS neurons that can fire at high frequencies may correspond to increased power in the 40+Hz oscillatory activity range in state 3, higher power in the 25 Hz range in state 2, and lower power oscillatory activity in state 1. Coordinating activity during experience dependent plasticity extends beyond cortex; FS regulation coordinates learning and memory in neural networks that excitatory cortical projection neurons participate in. Learning and memory includes but is not limited to FS oscillatory activity initiating new neurons born and integrating into neural networks in hippocampus, and may include any brain area connected directly or indirectly to cortical neurons.

Experience initiates FS maturation transitions and continued experience is required during transitions and to retain mature function. Disrupting FS neurons, for example though decreasing experience at the onset or during shifts in FS neuron experience dependent development, results in constitutively active cortical neural networks. In another example, activity is required during transitional states while FS neurons are encoding information; removing activity during transitions between state 1 and 2 may result in lethal epilepsy, as FS neurons may become arrested in an immature state, after FS neurons have retracted connections but prior to forming new connections. FS neuron dysfunctions may propagate widespread dysfunction, that may include and is not limited to the striatum, other cortical areas, and hippocampus. Removal of thalamic input to FS neurons results in unregulated biological neural networks that are constitutively active, meaning, excitatory neurons will fire even with weak or no thalamic input as excitatory neurons excite neighboring neurons and continue to receive input from other brain areas.

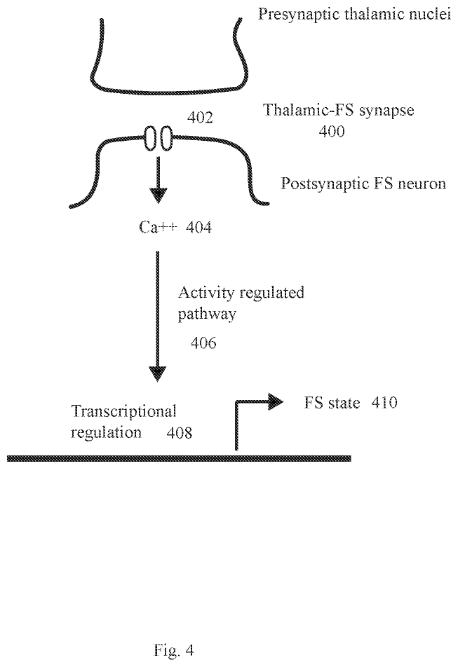

FIG. 4 depicts an illustration of activity dependent regulation of FS neuron states. Synaptic activity at thalamic-FS synapses (400) regulates activity dependent molecular pathways (406). Activity dependent molecular pathways may include and are not limited to molecular pathways that are regulated by calcium. For example, activation of NMDA receptors (402) at thalamic-FS synapses may increase available calcium (404), which regulates activity dependent molecular pathways (406), which transduce experience at thalamic-FS synapses to activity dependent transcriptional regulators (408). Activity dependent transcriptional regulators may include and is not limited to myocyte enhancer factor-2 c, a, and b, cAMP response element-binding protein, nuclear receptor related-1 protein, ubiquitin ligases and other small regulatory molecules, and the chromatin remodeling proteins methyl CpG binding protein 2 (MeCP2), fragile X mental retardation protein, histone deacetylase 1, ASH2-like protein, and the chromatin remodeling regulator bromodomain adjacent to zinc finger domain, 1B.

Activity dependent transduction of experience at thalamic-FS synapses regulates transcriptional regulation. FS maturation states may be identified by transcription factors in a similar way that cells are fated to different cell types during development. Transcription factors (408) that may regulate gene expression in FS neurons may include and are not limited to LEVI homeobox protein 6, visual system homeobox 2, TGFB-induced factor homeobox 2, zinc finger homeodomain 4, zinc fingers and homeoboxes 1, which are expressed in earlier FS maturation states, and homeobox C13, distal-less homeobox 3 (Hoxc3), alpha AT-hook transcription factor, forkhead box protein M1, ash2 (absent, small, or homeotic)-like (Drosophila) which are expressed in later FS maturation states. FS neurons may release growth factors and other small regulatory molecules that may change local cortical structures, for example, transforming growth factor beta 1, hepatoma-derived growth factor, vascular endothelial growth factor B, fibroblast growth factor 5, and brain derived neurotropic factor. Transcription factors enable the expression of genes that conifer FS functional properties in each maturation stage (410).

Disrupting experience disrupts regulation of activity dependent transcriptional regulation. Similarly, genetic variations in FS neural regulation of experience dependent plasticity may disrupt experience dependent shifts in FS regulation and myriad genetic variations are correlated to myriad neurological disorders, including but not limited to epilepsy, migraines, autism spectrum disorders, depression, anxiety, bipolar disorder, schizophrenia, intellectual disability, and neurodegenerative disorders. Genetic variation that underly disorders may conifer cognitive benefits, for example, variations that are linked to high intelligence are also linked to increased vulnerability to neurodegenerative disorders. De novo mutations have been correlated to the increase in incidence of neurological disorders, with de novo mutations accounting for the majority of vulnerability to myriad genetic disorders. As a highly metabolically active cell type, FS neurons are likely to be vulnerable to accumulation of de novo mutations. In support of this, differential expression of genes that are involved in regulating experience dependent plasticity are correlated to neurological disorders.

Selectively targeting FS neurons with therapeutic agents does not require individually modulating all of the downstream neurons reflecting dysfunction in FS regulation. Therapeutic agents may include and is not limited to providing experience, pharmacological agents, genome editing, stimulation of neurons, and exogenous treatment that otherwise seeks to modify or mimic FS neuron function. Selectively targeting FS neurons may include providing therapeutic agents for FS neuron dysfunction caused by disruptions in experience, including and not limited to early childhood trauma, deprivation, and traumatic events. For example, neglect in early childhood may appear as autism spectrum disorder.

In another aspect, selectively targeting FS neurons may be specified further by differentiating between genetic variations in activity dependent regulation of FS neurons shifting states and genetic variations in the genes expressed in each state. The functional properties of FS neurons in each state and FS regulation of neural networks in relation to experience may be elucidated by identifying FS neurons and quantifying properties of FS neurons. For example, a gene variant in Kv1.1 would be predicted to be reflected in FS neuron excitability and threshold to firing an action potential. Kv1.1 exhibits greater expression in more mature FS states, and identifying FS states may be used to distinguish between a genetic variant in Kv1.1 and state related changes in Kv1.1 expression. In another example, a loss of function mutation in the repressor MeCP2 would be predicted to correlate with precocious maturation of FS neurons. Differing genetic variants may have disparate influence on FS neuron, for example in the neurological disorder Rett Syndrome loss and gain of function mutations in MeCP2 may influence FS neuron state changes in disparate directions in patients who exhibit similar symptoms. Variations in transduction may be identified by the speed and activity required to shift FS neurons between states and FS neurons to retain states. In this example, genetic variations may underly the experience required to shift between states, with a loss of function mutation in the repressor MeCP2 being predicted to enable FS neurons to shift to more mature states with less experience and a gain of function mutation in MeCP2 that increases repression to require more experience to shift to a more mature state.

In another aspect, selective targeting of FS neurons enables conditional targeting. Exogenous genetic modifications may be environmentally regulated by including a promoter for transcriptional regulators expressed in a desired FS state. For example, a genetic modification in a novel AMPA receptor subunit may be restricted to expression in older states by including a promotor region for Hoxc13.

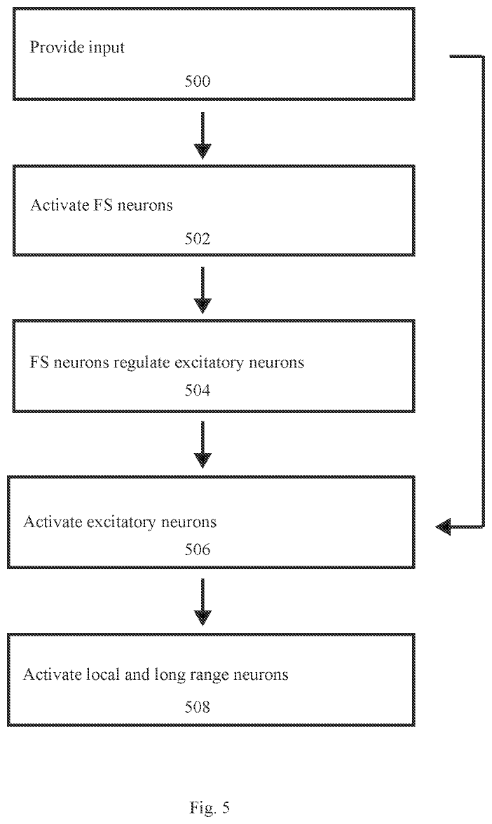

In reference to FIG. 5, a method of establishing a model of FS neurons regulating neural networks may be comprised of instantiating the model on one or more computer processors, by providing input to the neural network (500), activating FS neurons (502) which receive input, FS neurons regulating the activity of connected excitatory neurons (504), activating excitatory neurons in response to input (506), and activating local and long range neurons (508) in the network, defining connectivity properties based on biological neural networks in full or in part. It should be understood that a processor may be comprised of other processors or a combination of central processing units (CPUs), graphical processing units (GPUs), and other processors.

In biological neural networks, connectivity may be generally defined by connecting experience input that retains spatial and temporal properties to a plurality of FS neurons and weaker input to a comparatively larger plurality of excitatory neurons, locally connecting FS neurons to comparatively greater number of excitatory neurons in the network, and connecting excitatory neurons to a plurality of neurons in the network.

In reference to FIG. 6, a method of establishing a model of FS neurons regulating experience dependent plasticity in neural networks may be comprised of instantiating the model on one or more computer processors, by providing input (600) to the neural network, activating one or more FS neurons (602) which receive input, selectively changing or maintaining states in one or more FS neurons (604), FS neurons regulating the activity of a plurality of connected excitatory neurons (606), activating a plurality of excitatory neurons in response to input (608), and activating local and long range neurons in the network (610), defining connectivity properties based on biological neural networks in full or in part.

In biological neural networks, connectivity may be generally defined by connecting experience input that retains spatial and temporal properties to a plurality of FS neurons and weaker input to a comparatively larger plurality of excitatory neurons, locally connecting FS neurons to comparatively greater number of excitatory neurons in the network, connecting excitatory neurons to a plurality of neurons in the network, and selectively changing FS neuron properties and connectivity according to maturation state dynamics.

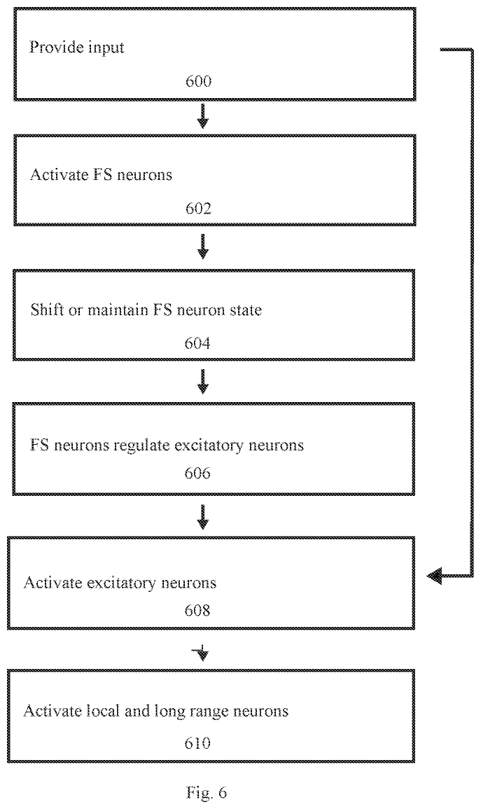

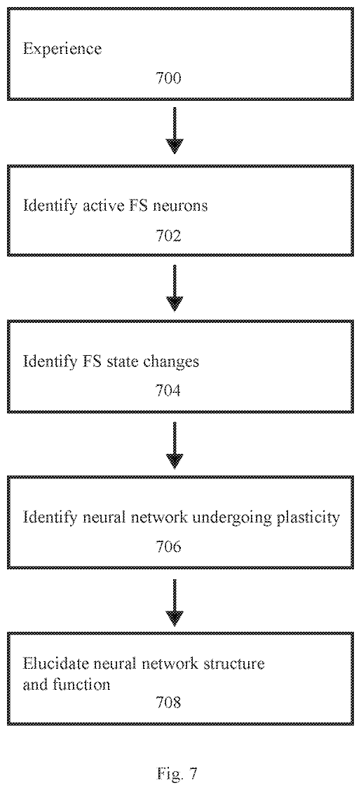

In reference to FIG. 7, a model of FS neurons regulating neural networks may be integrated with biological neural networks in order to elucidate the structure and function of neural networks that are active in response to experience, and more broadly higher cognitive tasks. Experience is provided as input to FS neurons through thalamic input (700). Thalamic input to FS neurons reflects the environment, meaning, the location of active FS neurons is representative of the neural networks receiving thalamic information originating from experience. FS activity (702) may be used to identify FS neurons and identify which neural network is active in response to an experience. For example, high frequency firing may be used to identify active FS neurons in a neural network. FS maturational states may be used to identify FS state changes (704), which regulate plasticity and regulatory properties in neural networks in response to experience. For example, changes over time in high frequency firing rate and firing properties or transitional states would indicate a shift in FS states.

Experience is reflected in the structure and function of neural networks. Identification of which neural networks are reflecting new experiences or optimizing experiences (706), through identification of properties of FS regulation of the network enables the elucidation of neural network structure and function (708). Identifying regulation of experience dependent plasticity through the timing of FS maturation changes may be used to elucidate which experience is encoded into a neural network during FS maturation state shifts. Local neural networks being regulated by FS neurons receive similar environmental input and the connections between of local neural networks and long range neurons may be identified through coordinated activity or comparable methods of identifying neural network connectivity. Neural network function and structure in relation to what was experienced during experience dependent plasticity may be used to understand the structure and function of the neural network that confers perception of an experience, which may be used to elucidate memories of experience and understand cognitive higher level thinking as the neural network is used at later times.

Elucidating the structure and function of neural networks that conifer a specific experience and cognitive function may be used to inform applications that require understanding of biological neural networks, including and not limited to the design of AI informed by neural networks and predictions of neural network structure and function. Myriad techniques may be combined with FS shifts and the structure and function of neural networks, including and not limited to next generation brain recording techniques and fine scale brain mapping techniques.

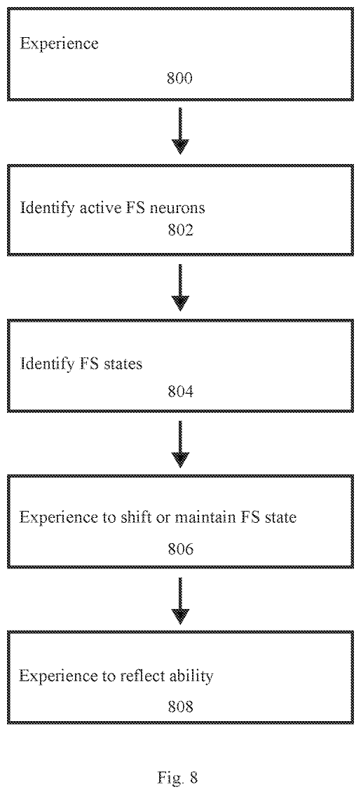

In reference to FIG. 8, a model of FS neurons regulating experience dependent plasticity in neural networks may be integrated with biological neural networks in order to shift neural networks to reflect desired cognitive abilities. FS regulation of neural networks in relation to experience (800) may be elucidated by identifying maturation states (804) in active FS neurons (802). Experience then can be provided to shift FS neurons (806) in a desired way. For example, novel experiences initiate FS neuron shifts into less mature states in many individuals. Experience may then be provided while FS neurons are shifting (808), enables the neural network to encode new abilities. Maintaining maturation similarly may be used to optimize (808) existing neural networks by providing experience (806) that retains mature states in the FS neurons that regulate a neural network of interest.

Predictions regarding the type of experience required to conifer specific cognitive abilities may be performed using AI or comparable predictive method. As would be known by a skilled practitioner, myriad predictions may be performed using data relating to experience and neural networks. AI based predictions, for example may be used to design therapeutic agents that enhance or ameliorate FS functional properties for a specific FS maturation state or cognitive function. For example, generation of virtual experiences by AI may be combined with the model in this disclosure in real time or through predictions. In another example, the model may be combined with AI predictions applied to the design of novel pharmacological agents.

In another aspect, integrating biological neural networks with a model of FS neurons regulating experience dependent plasticity in neural networks may be used to provide recommendations for experience, including and not limited to recommendations that reflect individual genetic variations and individual preferences. Similarly, recommendations may be provided for administration of pharmacological agents that may influence FS function in a specific FS maturation state or FS transitional state.

In reference to FIG. 9, a method of establishing a model of FS neurons regulating neural networks integrated with biological neural networks in order to elucidate neural network structure and function may be comprised of instantiating the model on one or more computer processors, by recording neurons and experience (900), identifying activity in FS neurons (902), identifying activity in neural networks regulated by FS neurons (904), identifying FS maturation states (906), and elucidating the structure and function of one of more neural networks (910) in relation to experience.

In another aspect, a method of establishing a model of FS neurons regulating neural networks integrated with biological neural networks in order to initiate experience dependent plasticity may be comprised of instantiating the model on one or more computer processors, by recording neurons and experience (900), identifying one or more active FS neurons (902), identifying the FS neuron state (904) in one or more FS neurons, providing experience (908) to change or maintain one or more FS states, and providing experience to elucidate (910) one or more neural networks and encode abilities into the network (912).

In the preferred embodiment, FS neurons are recorded (902) during experience (900) using an electroencephalogram (EEG) device, however, it should be understood by one skilled in the art that myriad neural recording methods may be used. In the preferred embodiment, FS neuron firing properties may be identified (902) by selecting high frequency firing activity, which may be defined as oscillatory activity in the 20 Hz-100+ Hz frequency range. Spatial information from the location of EEG sensors may be used to locate active neural network(s) receiving thalamic input by identifying high frequency activity. Neural networks that include connections to local neurons and neurons in deeper brain areas (904) may be identified through coordinated FS activity and relevant markers, for example, hippocampal ripples or coherence of gamma oscillations in other cortical areas. High frequency oscillatory brain activity, and other frequencies of brain activity in neural networks, may be analyzed according to methods of EEG data analysis known to one skilled in the art, for example high-pass filtering data, applying a fast Fourier transform, and predicting power spectral density for a frequency or frequency range. Maturation states (906) may be identified by the population of FS neurons that are capable of firing at high enough frequencies to initiate oscillatory activity, with the frequency corresponding the age of each state, or a comparable method. Biological neural network and environmental data may be acquired and analyzed on a processor and stored on memory. Experience may be recorded by a camera, a virtual or augmented reality, user generated input, sensors, tracking connected technologies, a user interface, or other comparable method.

In the preferred embodiment, recommendations (912) may be provided to a user by connecting a brain recording wearable to a user interface. A brain recording wearable device may include a touch screen, camera, microphone and speaker, or combination therein to enable a user interface. A user interface may include and is not limited to a voice assistant, text based interface, graphical interface, or a combination therein.

In another aspect, a brain recording wearable device may be connected across technologies using wireless technology. Recommendations (912) provided to a user may be integrated with other forms of digital mediums that may be generated using AI or a comparable method, including and not limited to music, virtual reality experiences, augmented reality experiences, and digital media. Recommendations for drug agents or gene editing may be provided through myriad methods, for example, and AI based voice assistant or clinical test. Recommendations may include and are not limited to prescribing therapeutic agents, design of a new drug agent, personalized medicine, gene editing, remediating environmental pollutants that my lead to an increase in de novo mutations, including and not limited to the design of bacteria or plants that reduce environmental pollutants that contribute to mutations.

In one aspect, data may be classified using AI or an analogous method to form a database of neural data in relation to experience. Data may be classified according to myriad properties, for example, according to the experience properties, FS neuron properties and FS neuron state, and the spatial location of FS neurons, and the activity of neurons being regulated by FS neurons. FS neurons are spatially organized into a neural network comprised of FS neurons regulating neural network function in a way that reflects experience and higher cognitive abilities. Classifications of data may be instantiated on a processor and stored on memory.

As would be understood by one skilled in the art, a brain recording wearable containing one or more processors is an example of a method and myriad methods may be used. It should be understood that FS neurons may be identified using myriad methods, in part or fully, including but not limited to functional near-infrared spectroscopy, functional magnetic resonance imaging, ultrasonic brain stimulation and recording devices, transcranial electrical or magnetic stimulation devices, next generation neuroscience recording and simulation techniques for example nanotechnologies and optogenetics, brain implants, genomics techniques, proteomics techniques, gene markers, genome sequencing, brain mapping techniques, extracellular brain recording techniques, intracellular brain recording techniques, pharmacological agents, and methods indirectly correlated to biological data for example internet usage and text analysis. Modifications and this invention may include any of these additional methods.

Therapeutic agents similarly may be designed to target FS neurons using myriad methods as would be known by one skilled in the art. For example, genetic variations for an individual may be identified by performing whole exon sequencing using next generation genome sequencing and comparing the resulting sequence to a reference genome. Clinically relevant mutations may be identified by quantifying the anticipated mutation rate to identify mutations that occur with frequency significantly above expected variation and identification of variations that are probabilistically likely have clinical relevance. Drug treatments and gene editing sequences may be assayed by quantifying FS neuron function in cells engineered to contain mutations of interest or comparable methods. A gene editing technique may be used to insert a gene variation or a combination of mutations into cultured cortical neurons or an analogous method. Cortical neurons can be examined using electrophysiology techniques to assess the role of a gene variant on FS neuron function; connectivity and firing properties can be assed through recording FS neurons using electrophysiology methods. Activity can be modulated by adding Ca++ to the culture. New drug treatments can be assayed by applying drug candidates to the culture bath and similarly assaying FS function. A genetic modification may include a sequence for a bioluminescent molecule, including but not limited to green fluorescent protein, allowing a modification to serve as a sensor for a state of interest or environmental regulation of states.

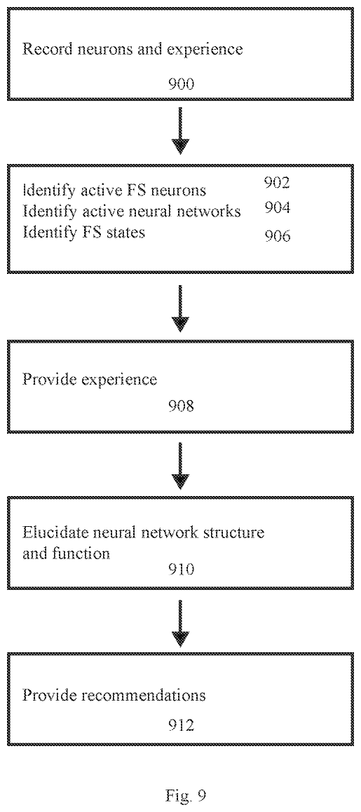

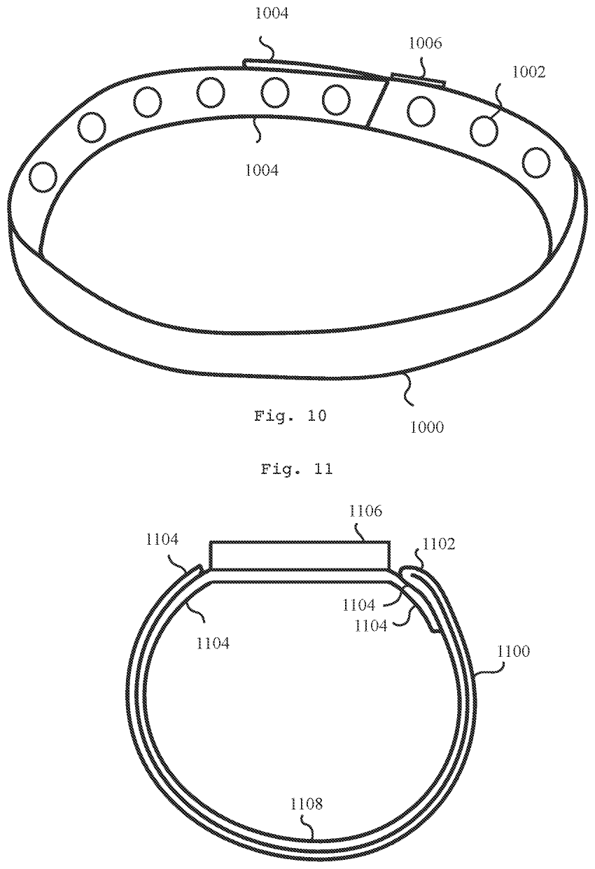

A representative example of a brain recording wearable that is an electroencephalogram (EEG) device is shown in FIG. 10. The brain recording wearable may be comprised of a band (1000) made of four-way stretch cotton blend fabric containing a plurality of sensors (1002), that may be fastened (1004) around the head as a headband in a position that corresponds to sensors spatially localized to sense activity in prefrontal, temporal, visual, and parietal cortex. The sensors may be changed in configuration by changing positioning within the band, with alterations in sensor number and positions corresponding to recordings of populations of neurons in different brain areas. In this example, sensors are comprised of four-way stretch woven silver coated fabric with <2 Ohm resistance, insulated with a polyurethane coating. Sensors are connected to an integrated circuit for neural recordings, with insulated silver coated conductive leads with comparable resistance to the sensors. A reference sensor is connected with the similar leads. The integrated circuit for neural recordings and associated support hardware are contained in a housing (1006). The sensors may be sewed into an inner layer of the band, however, myriad methods may be used to attach sensors into a band, including and not limited to a polymer glue, interlocking pieces, fabric welding or a combination therein.

A band comprised of fabric that stretches or comparable material enables users with sensory sensitivity to wear a brain recording wearable. A band may be comprised of myriad materials that would be known by a skilled practitioner, including but not limited to fabrics, plastics, synthetic polymers, woven fabrics, and a combination therein. Similarly, myriad brain recording sensors may be used. In the example provided, the headband is fastened around the head (1004) using velcro or an analogous method, and as would be known by a skilled practitioner the band may be fastened by myriad methods including and not limited to one or more buttons, magnetic fasteners, interlocking pieces, snaps, hooks, or may be one continuous piece of fabric with higher stretch in one or more places, or a combination therein. In the example shown, the fastener is sewn or otherwise attached to the outer layers of fabric on each end, which is made slightly longer than the inner layer containing sensors, such that the fastener is not in direct contact with the user when the band is in use. The band may optionally include additional fabric to cover sensors while not in use.

Referring to FIG. 11, the brain recording wearable (1100) band containing a plurality of sensors (1108) may be adjusted in length to be optionally worn on the user's wrist. In the example provided, the brain recording wearable band is looped (1102) and may be fastened (1104) using velcro or an analogous method. In this example, the length is adjusted by fastening one side to velcro or an analogous way to fasten, and then wrapping the band around the wrist twice and fastening the other side of the band. The band may be adjusted to fit around the wrist or a similar appanage, for example the upper arm. Adjusting length enables a band to be worn around the wrist or the head. This enables the user to easily carry the brain recoding wearable even when not actively recording their brains. The representative example shown enables optionally adding or modifying sensors to include additional measurements, including and not limited to measuring heart rate. A brain recording wearable band may be modified to be worn on the wrist using myriad methods, including and not limited to looping the band through one or more connectors, securing excess fabric using a watch band holder, modifying the degree of band stretch. The example provided includes a housing (1106) that may contain a components to enable a user interface, such as a touchscreen, camera, microphone and speaker, and associated hardware.

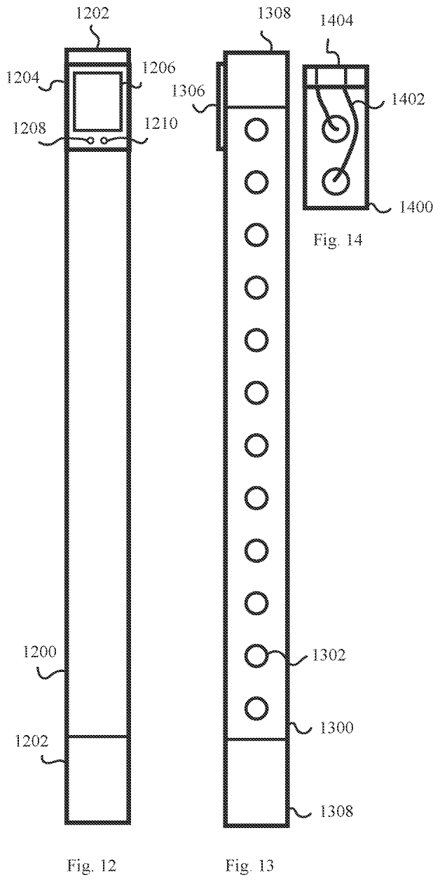

An outer view of the brain recording wearable is shown in FIG. 12, illustrating the placement of the fasteners on each end (1202) of the band (1200), a housing (1204), a touchscreen (1206), a microphone (1208), and a camera (1210). FIG. 13 shows an inner view of the brain recording wearable, which is the side that contacts the skin of the user, illustrating the placement of the fasteners on each end (1308) of the band (1300), a housing (1306), and sensors (1302). FIG. 14 shows a view of the inside of the band (1400) to show the attachment of sensor leads (1402) to an integrated circuit for neural recordings (1404).

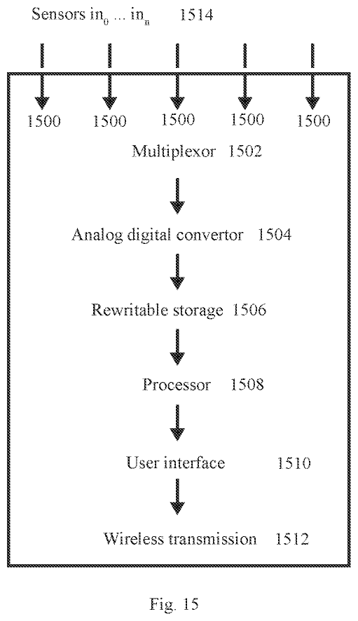

A simplified circuit diagram is provided in FIG. 15. An integrated circuit for neural recordings may be comprised of sensor inputs (1514), signal amplifiers (gain) (1500), a multiplexer that parallelizes signals (1502), an analog digital converter (1504), and memory (1506) and may include a processor (1508), for example a dual 8 core 32-bit ARM processor. A brain recording wearable device may be enabled to connect across technologies by including a user interface (1510), and wireless transmission of data (1512). A brain recording wearable may include a microphone and a speaker to enable a voice assistant, and a touchscreen interface, wireless internet, bluetooth, or cellular radio frequency, battery, one or more cameras, GPS, an accelerometer, and may contain peripheral sensors, for example a heart rate sensor, or a combination therein. Components are shown contained in one housing, however, they may be contained in a plurality of housings. Similarly, one or more components in an integrated circuit may be connected in an alternative manner.

Brain activity and environmental data may be processed using a remote GPU, CPU, or the processor contained in the EEG wearable device, and stored on a machine-readable storage device, a machine-readable storage substrate, a random or serial access memory device, or a combination of these methods. A device may connect to a mobile device, a tablet, laptop, desktop computer, television, projector, virtual or augmented reality device, or any voice-activated connected technology, allowing a voice assistant to access AI generated by the neuroscience device across technologies.

It should be understood that a brain recording wearable device that may be worn around the wrist may be modified to include comparable methods of recording brain activity and additional types of sensors, including and not limited to next generation neuroscience recording and simulation techniques for example functional near-infrared spectroscopy, functional magnetic resonance imaging, ultrasonic brain stimulation and recording devices, transcranial electrical or magnetic stimulation devices, devices based on activating channel rhodopsin, or a combination therein.

Although the invention in this disclosure has been described with reference to specific embodiments, myriad modifications thereof will be apparent to one skilled in the art without departing from the spirit and scope of the invention.

The invention claimed is:

1. A system of modeling neural networks wherein FS neurons regulate the neural network, comprised of:
a plurality of neurons forming a neural network, wherein a plurality of FS neurons and a plurality of excitatory neurons receive input originating from the environment, FS neurons are connected to more neurons and with greater weight as compared to excitatory neurons, FS neurons are active in response to input originating from the environment in advance of excitatory neurons, FS neurons regulate excitatory neurons by inhibiting activity in a plurality of excitatory neurons.

2. The system of claim 1, wherein FS neurons regulate which neurons are active.

3. The system of claim 1, wherein FS neurons regulate plasticity in neural networks by coordinating activity.

4. The system of claim 1 wherein FS neurons regulate experience dependent plasticity in neural networks by changing states.

5. The system in claim 4 wherein experience dependent plasticity may be used to encode new abilities.

6. The system in claim 4 wherein experience dependent plasticity may be used to elucidate the structure and function of neural networks.

7. The system in claim 1 wherein FS neurons are selectively targeted by therapeutic agents.

8. One or more non-transitory computer readable media storing instructions that when executed by one or more processors cause the one or more processors to implement a model of a neural network, the model neural network comprising: a plurality of neurons forming a neural network, wherein a plurality of FS neurons and a plurality of excitatory neurons receive input originating from the environment, FS neurons are connected to more neurons and with greater weight as compared to excitatory neurons, FS neurons are active in response to input originating from the environment in advance of excitatory neurons, FS neurons regulate excitatory neurons by inhibiting activity in a plurality of excitatory neurons.

9. The method of claim 8, wherein FS neurons regulate which neurons are active.

10. The method of claim 8, wherein FS neurons regulate plasticity in neural networks by coordinating activity.

11. The method of claim 8 wherein FS neurons regulate experience dependent plasticity in neural networks by changing states.

12. The method in claim 11 wherein experience dependent plasticity may be used to encode new abilities.

13. The method in claim 12 wherein a neural recording device may record and model biological data.

14. The method in claim 13 wherein a neural recording device may provide recommendations.

15. The method in claim 11 wherein experience dependent plasticity may be used to elucidate the structure and function of neural networks.

16. The method in claim 15 wherein a neural recording device may record and model biological data.

17. The method in claim 8 wherein FS neurons are selectively targeted by therapeutic agents.

18. A brain recording wearable, the brain recording wearable comprising: a band, a plurality of brain recording sensors, a fastener, wherein the band may be fastened around the head, wherein the band length may be adjusted and fastened around an appendage.

19. wherein the band contains an integrated circuit for neural recordings.

20. wherein the band contains components enabling a user interface.
